(19) United States
(12) Patent Application Publication
(10) Pub. No.: US 2018/0024074 A1
(43) Pub. Date: Jan. 25, 2018

(54) DEVICES AND METHODS FOR DETECTION OF COUNTERFEIT OR ADULTERATED PRODUCTS AND/OR PACKAGING

(71) Applicant: THE UNITED STATES OF AMERICA, AS REPRESENTED BY THE SECRETARY, DEPARTMENT OF HEALTH & HUMAN SERVIC, Silver Spring, MD (US)

(72) Inventors: Nicola Ranieri, Maineville, OH (US); Ja Cinta Shaques Batson, Cincinnati, OH (US); Stefanie Lee Kremer, Fort Thomas, KY (US); Stanley Frank Platek, Independence, KY (US); Mosley Fulcher, Liberty Township, OH (US); Mark R. Witkowski, West Chester, OH (US); Robert Duane Satzger, Cincinnati, OH (US)

(73) Assignee: THE UNITED STATES OF AMERICA, AS REPRESENTED BY THE SECRETARY, DEPARTMENT OF HEALTH & HUMAN SERVIC, Silver Spring, MD (US)

(21) Appl. No.: 15/547,416

(22) PCT Filed: Feb. 1, 2016
(86) PCT No.: PCT/US2016/016019

§ 371 Date: Jul. 28, 2017

Related U.S. Application Data
(60) Provisional application No. 62/110,090, filed on Jan. 30, 2015.

(51) Int. Cl. G01N 21/88; G01N 21/25; G06K 9/20; G06T 7/00; G06K 9/62; G01N 21/95; G01N 33/02

(52) U.S. Cl. CPC G01N 21/8806; G01N 21/9508; G01N 21/255; G01N 33/02; G06T 7/001; G06K 9/6202; G06K 9/209; G06K 9/2027; G06K 9/2018; G01N 2201/0221; G01N 2201/0627; G01N 2021/8845; G01N 2021/1748

(57) ABSTRACT

Disclosed are devices and methods for detecting a counterfeit or adulterated products and/or packaging. The device (20) includes a plurality of light sources (28) configured to emit light at a plurality of different wavelengths onto an object, at least two image acquisition devices (34a, 34b) adapted to acquire first and second image data, and first and second imaging displays (36A, 36B) configured to display the first and the second image data, respectively.

### CROSS-REFERENCE TO RELATED APPLICATIONS

The present application claims priority to U.S. Provisional Application No. 62/110,090, filed on Jan. 30, 2015, entitled Device and Method for Detection of Counterfeit Pharmaceuticals and/or Drug Packaging. The contents of this application are incorporated by reference in its entirety.

### STATEMENT REGARDING GOVERNMENTAL SUPPORT

The present subject matter was made with U.S. government support. The U.S. government has certain rights in this subject matter.

### FIELD OF INVENTION

The present invention generally relates to methods and devices for the detection of counterfeit or adulterated products, such as pharmaceuticals, and/or the detection of counterfeit packaging, and more particularly to devices and methods for detection of counterfeit or adulterated products and/or the packaging using visible and non-visible radiation and still more particularly to devices and methods for in-situ detection of counterfeit or adulterated products (e.g. pharmaceuticals) using visible and non-visible radiation. Although a preferred use of the devices and methods according to the inventive concepts disclosed herein is for the detection of counterfeit pharmaceuticals or drug packaging, the devices and methods disclosed herein can also be used in a variety of other forensic and public health applications, such as for example, the detection of adulterated extra virgin olive oil, filth analysis (e.g. insect parts detection in rice), forensic crime scene analysis and evidence collection, sexual assault examination and body fluid searches.

### BACKGROUND OF THE INVENTION

The amount of counterfeit pharmaceuticals entering the United States continues to increase. Such counterfeit pharmaceuticals are illegally imported into the United States, and are commonly available over the Internet. It may be difficult to determine the authenticity of a pharmaceutical, since the genuine and counterfeit products may have nearly identical appearances and markings (e.g., shape, color, size, packaging, labeling, etc.), even when viewed by professionals. The detection of counterfeit pharmaceuticals is important, in part because the efficacy of a counterfeit product may be lower than that of the actual product. In addition, a counterfeit product may contain toxic components or components that might cause side effects which are not associated with the authentic product. Also, the illegal sale of counterfeit products results in severe monetary loss to pharmaceutical companies and retailers.

Current methods for detecting counterfeit pharmaceuticals include vibrational spectroscopy, x-ray diffraction, gas chromatography, liquid chromatography and mass spectrometry. These methods, although often effective, require expensive and bulky instrumentation, and are generally performed in a laboratory.

It thus would be desirable to provide a new device and methods for detecting counterfeit pharmaceuticals and/or packaging for the pharmaceuticals and/or packaging from an authorized manufacturer, supplier and the like. It would be particularly desirable to provide such devices and methods that would be portable and usable at any desired location such as the inspection point for customs. It also would be particularly desirable to provide such devices that would be hand-held and use visible and/or non-visible light to illuminate suspect pharmaceuticals and/or packaging and determining from such illumination if the pharmaceuticals and/or packaging being examined are counterfeit pharmaceuticals and/or packaging. Such detection devices preferably would be simple in construction and less costly than prior art devices and such methods would not require highly skilled users to utilize the device.

### SUMMARY OF THE INVENTION

One aspect of the inventive concepts disclosed herein provides a device for detecting a counterfeit product. The device includes, inter alia, a plurality of light sources configured to emit light at a plurality of different wavelengths onto an object; at least two image acquisition devices adapted to acquire first and second image data; and first and second imaging displays configured to display the first and the second image data, respectively.

This aspect of the invention can have a variety of embodiments. In some embodiments, the plurality of light sources include light emitting diodes, e.g., single-wavelength light emitting diodes. According to aspects, the first image acquisition device is adapted to detect near-infra-red light and the second image acquisition device is adapted to detect light in the visible to ultraviolet spectrum. According to aspects, the at least two image acquisition device each include one or more CCD arrays.

The device can further include a housing, wherein the plurality of light sources and the at least two image acquisition devices are coupled to the housing so that the plurality of light sources and the at least two image acquisition devices are maintained in fixed relation to each other while the object is being illuminated.

The device may further include a control device adapted and configured to control operation of the plurality of light sources and the at least two image acquisition device and according to aspects, the control device is adapted to operate white light sources to flash sequentially. The device can include one or more filters adapted to selectively condition light entering the at least one image acquisition device.

The at least one image acquisition device can include one or more CCD arrays.

At least one of the light sources of the plurality of light sources is adapted to emit light having an infrared wavelength. At least one of the light sources of the plurality of light sources is configured so as to emit light having a visible wavelength. At least one of the light sources of the plurality of light sources is configured to emit light having an ultraviolet wavelength. The light sources can be LED light sources. In certain instances the LEDs used can be thermally cooled with higher lumen/W output as compared to conventional LEDS. The light sources can be tungsten light sources. According to aspects, the plurality of light sources includes at least 12 light sources, each light source adapted to emit light of a wavelength different from the wavelengths emitted by other light sources.

According to aspects of the inventive concepts, the device further includes at least three legs adapted to allow for stationary operation of the device. According to other aspects, devices may include four or more legs. The legs can be adapted to have adjustable lengths and the legs can be adapted to twist on and off. And according to further aspects, the device can have a light shield adapted to prevent stray light from illuminating the object, the shielded light may be in the infrared band and the shield includes at least one magnet adapted to magnetically attach the shield to the body of the device. Also, a plurality of magnets may be used to magnetically attach the shield. The device can have video in and video out ports, and/or image storage capabilities.

Another aspect of the invention provides a method for detecting a counterfeit product, including providing a device according to claim 1; imaging, on each of the first and the second imaging displays, a product using first and second lighting schemes, respectively; and comparing the images on the first and the second imaging displays to one of images of analogous authentic products and images of counterfeit analogous products, wherein the compared images were created using the first and second lighting schemes, respectively.

### BRIEF DESCRIPTION OF THE DRAWING

So that those having ordinary skill in the art to which the present disclosure pertains will more readily understand how to employ the systems, devices and methods of the present disclosure, embodiments thereof will be described in detail hereinbelow with reference to the drawings, wherein:

FIG. 1 is a perspective view illustrating the basic principle used in some advantageous embodiments of the inventive concepts;

FIG. 2A is a schematic diagram of an embodiment of a counterfeit drug-detecting LED device of the present disclosure;

FIG. 2B is a schematic diagram of another embodiment of a counterfeit drug-detecting LED device of the present disclosure;

FIG. 3A is a top view of one embodiment of a counterfeit drug-detecting LED device of the present disclosure;

FIG. 3B is a bottom view of one embodiment of the counterfeit drug-detecting LED device of the present disclosure;

FIG. 4 is a perspective view showing an embodiment of the counterfeit drug-detecting LED device being used to inspect two tablets;

FIG. 5 is a perspective view showing an embodiment of the counterfeit drug-detecting LED device being used to inspect tablets in their packaging;

FIG. 6A provides a high level flow diagram illustrating an embodiment of the methodology of the present disclosure;

FIG. 6B provides a further high level flow diagram illustrating an embodiment of the methodology of the present disclosure;

FIG. 7 depicts tilted image acquisition devices according to an embodiment of the inventive concepts;

FIG. 8 depicts a menu screen according to an embodiment of the inventive concepts;

FIG. 9 depicts a microcontroller configured to control electronic components according to an embodiment of the present inventive concepts;

FIG. 10 depicts illustrative CCD locations and angles according to embodiments of the inventive concepts;

FIG. 11 depicts a device including legs that can be extended to allow the device to stand on a table, desk, counter, or other surface according to an embodiment of the inventive concepts;

FIG. 12 depicts a device including removable legs that can be used to allow the device to stand on a table, desk, counter, or other surface according to an embodiment of the inventive concepts and be removed for convenience; and

FIG. 13 provides a series of photographs illustrating a field screening using a device of the present disclosure of counterfeit and authentic product cartons, bottles and capsules.

These and other aspects of the subject disclosure will become more readily apparent to those having ordinary skill in the art from the following detailed description of the invention taken in conjunction with the drawings.

### DETAILED DESCRIPTION OF PREFERRED EMBODIMENTS

Disclosed herein are detailed descriptions of specific embodiments of devices, systems, apparatus and methods for the detection of counterfeit or adulterated products and/or packaging. It will be understood that the disclosed embodiments are merely examples of the way in which certain aspects of the invention can be implemented and do not represent an exhaustive list of all of the ways the invention may be embodied. Indeed, it will be understood that the systems, devices and methods described herein may be embodied in various and alternative forms. Moreover, the figures are not necessarily to scale and some features may be exaggerated or minimized to show details of particular components.

Although a preferred use of the devices and methods according to the inventive concepts disclosed herein is for the detection of counterfeit pharmaceuticals or drug packaging, the devices and methods disclosed herein can also be used in a variety of other forensic and public health applications, such as for example, the detection of adulterated extra virgin olive oil, filth analysis (e.g. insect parts detection in rice), forensic crime scene analysis and evidence collection, sexual assault examination and body fluid searches.

Still further the presently disclosed device and methods have been tested for use in the following alternative applications:

| | |
|---|---|
| Label copying, alterations, | Fingerprints |
| version substitutions, etc. | |
| Covert/Hidden security features | Adhesives differences |
| in printing technology | |
| Pharmaceutical excipients | Tobacco packaging and paper |
| Tablet core and coating (90+ | Rat poison in dough |
| product library of finished dosage) | |
| Tablet homogeneity/blending | Sunglasses polarized/UV |
| Capsule shells and content | Crime scene investigation tool |
| Pharmaceutical product diversion | olive oil |
| Veterinary liquid meds. | juices |
| Dark colored liquids (i.e. soft | jewelry/gems |
| drinks, grape juice or jelly, etc.) | |
| Cosmetic product (i.e. makeup | Clothing |
| blushes and powders, chap stick, etc.) | |
| Document fraud (i.e. date or | |
| signature changing, etc.) | |
| ID cards counterfeiting | |

Well-known components, materials or methods are not necessarily described in great detail in order to avoid obscuring the present disclosure. Any specific structural and functional details disclosed herein are not to be interpreted as limiting, but merely as a basis for the claims and as a representative basis for teaching one skilled in the art to variously employ the disclosure systems, devices, apparatuses and methods.

The present disclosure now will be described more fully, but not all embodiments of the disclosure are necessarily shown. In addition, many modifications may be made to adapt a particular situation or material to the teachings of the disclosure without departing from the essential scope thereof.

The present invention in its broadest aspects includes devices and methods for the detection of suspected counterfeit pharmaceuticals including the packaging thereof. Such methods include exposing an authentic pharmaceutical and a corresponding suspected counterfeit pharmaceutical to one or more light sources having selected wavelengths and visually detecting a difference in color, brightness, contrast, darkening and/or other visual effect(s) between the authentic and suspected counterfeit pharmaceuticals. The device, embodying such methods, includes a plurality of light sources that generate the light to which the authentic and suspected counterfeit pharmaceuticals are exposed. In further embodiments, such light is generated using a hand-held, portable device and one or more LED (light emitting diode) comprise the one or more of the plurality of light sources.

As indicated above, the suspected counterfeit is visually observed when exposed to the light from the one or more light sources to determine if there is a difference in color and/or other visual effect(s), such as brightness, contrast, darkening, between an authentic pharmaceutical/packaging and the suspected counterfeit. Such differences occur because the light characteristics (e.g., light reflection, light absorption and fluorescence) are dependent upon the composition and makeup of the pharmaceutical and/or packaging. In other words, a difference in the composition or formulation between a counterfeit pharmaceutical and that for an authentic pharmaceutical can be revealed as a change in color and/or other visual effect(s) such as brightness, contrast, darkening, particularly when the two are exposed to different wavelengths of light and/or radiation. Similarly, differences in the materials used in the packaging components between the counterfeit and authentic packaging also should be visually observed when the two are exposed to different wavelengths of light/radiation.

It has been found that differences in color and/or other visual effect(s) such as brightness, contrast, darkening, are observable when authentic and counterfeit pharmaceutical products and/or product packaging are illuminated with appropriate wavelengths of light, and also when being observed through appropriate filters. Without being bound by any particular theory, it is believed that these differences in color and/or other visual effect(s) such as brightness, contrast, darkening, are produced by slight differences in the fluorescent response of the excipients (or other components) within the pharmaceutical product (e.g., tablet or capsule), in the inks on the products, or in the product packaging itself. It has also been noted that lot-to-lot variability in authentic pharmaceutical products in these properties are minimal since the production processes of such products are highly controlled. Thus, the appearance of different lots of such authentic pharmaceuticals will be very similar when viewed under different wavelengths of light. In contrast, suspect counterfeit products do not have a single source, are not controlled as highly in the various sources, and consequently have a greater variability in appearance and will generally appear different from authentic products and packaging. For example, a counterfeiter may use an ink or dye that has a different composition than the originator. Although this different ink or dye composition may appear to have the same color to the naked eye, the different composition may fluoresce differently.

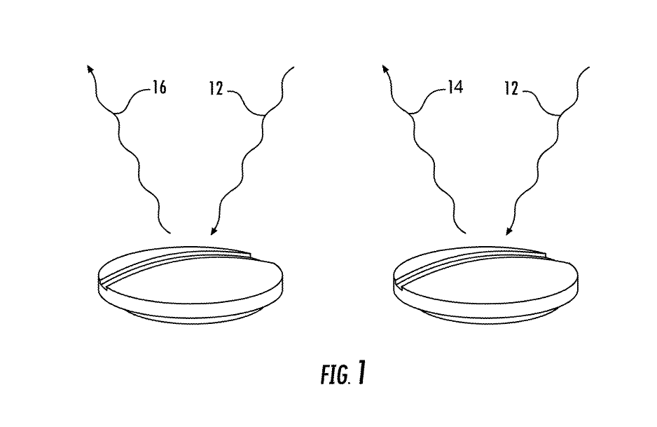

Referring now to the various figures of the drawing wherein like reference characters refer to like parts, a schematic diagram illustrating the basic principle used in advantageous embodiments of the invention is shown in FIG. 1. As schematically illustrated, the incident light 12 impinges upon both a counterfeit and authentic pharmaceutical product (depicted in the figure as a tablet).

The intensity and wavelengths of the reflected light 14, 16 differs between the two products, resulting in an observable difference in color and/or other visual effect(s) between the two products and/or the product packaging. This observable difference occurs upon illumination with light of one or more particular wavelengths, which results in an observable difference in color and/or other visual effect(s). Also, differences that are not detectable by visually inspecting a pharmaceutical product with the naked eye under ambient lighting conditions are detected using the device and methods described herein. This principle is utilized in embodiments of the invention to produce inexpensive and portable devices and screening methods for determining whether a product, such as a pharmaceutical, of unknown origin is legitimate or not or whether a product has been adulterated.

Exemplary Counterfeit Pharmaceutical Detection Devices

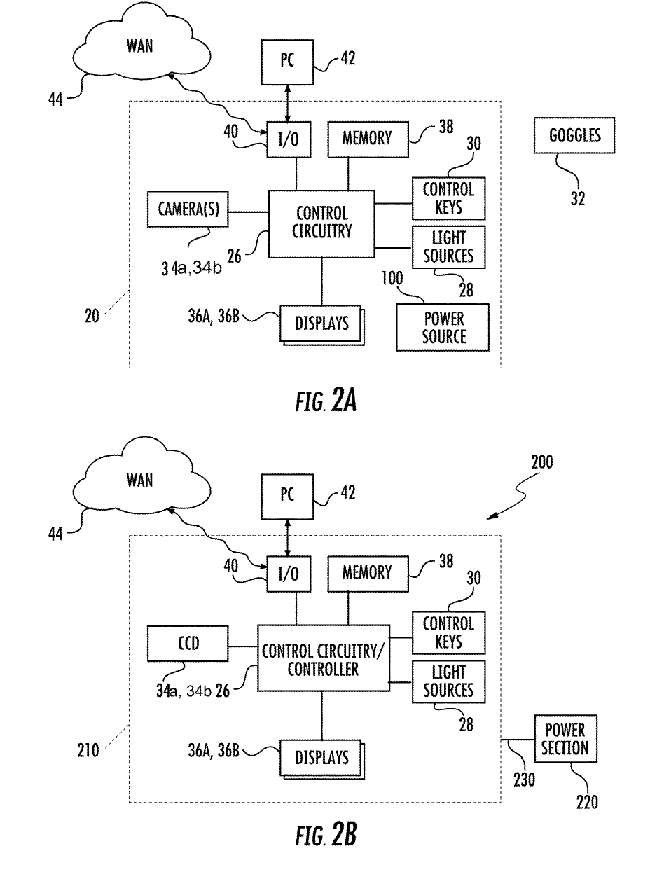

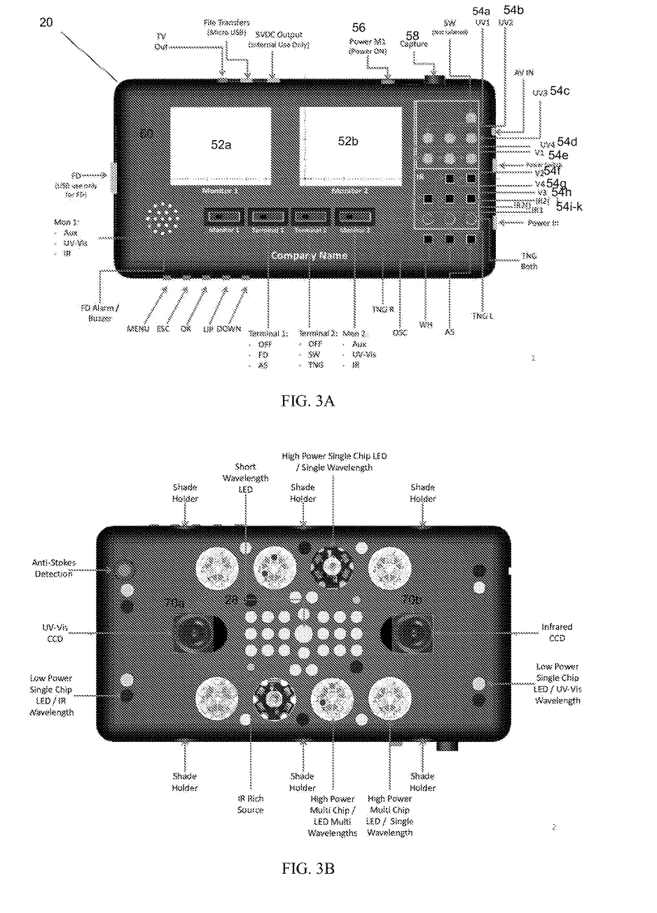

Referring now to FIGS. 2A and 2B, there is shown one schematic embodiment of a counterfeit pharmaceutical detection device 20 according to aspects of the present inventive concepts. Such a device 20 may include control circuitry 26 that is composed of circuit components or elements that can carry out the functions described herein and/or such components along with a controller (e.g., a microprocessor, microcontroller, application specific integrated circuit (ASIC) or the like) or a controller. Such control circuitry 26 may be configured and arranged so as to control the functionalities of the detection device including the light sources 28, display(s) 36a, 36b and at least two image acquisition device(s) such as a camera(s) 34a, 34b (FIG. 3B). The at least two image acquisition devices 34a, 34b may be to acquire first and second image data. Likewise, the first and second imaging displays 36a and 36b may be configured to display the first and the second image data, respectively.

In illustrative embodiments, such a device 20 also may also include a plurality of switches, buttons, or control keys 30 that are operably coupled to the control circuitry 26 that are used by the user for turning on or off one of more light sources 28. The switches, buttons, or control keys 30 can be implemented as physical hardware components and/or software elements (e.g., widgets displayed on a graphical user interface). Alternatively, the detection device 20 may embody any of a number of other devices or techniques as is known to those skilled in the art that can control the selection of the one or more light sources. In an illustrative embodiment, the detection device displays 36a, 36b may be configured so as to emulate a touch screen having for example one or more buttons displayed thereon each being representative of a light source. Thus, when a user touches one of the displayed buttons the control circuitry causes the corresponding light source to be turned on/off. In yet another embodiment, a touch pad may be provided that controls a cursor depicted on the screen. By moving the cursor to one of the buttons depicted on the screen and actuating the touch pad, the user can cause a given light source to be turned on/off.

In yet further embodiments, the control circuitry 26 may be configured so that when a user provides an input indicating that the detection device is appropriately positioned with respect to the suspect product, the control circuitry includes instructions and criteria that controls the selection of the light sources, the turning of the light sources on/off as well as the sequence and which light sources to turn on/off, and the acquisition of image data using the appropriate image acquisition devices.

As described herein, the light sources are configured and arranged so as to have particular wavelengths that are used for the illumination of a product and/or product packaging. As described herein, the illumination of the products, such as pharmaceuticals under specific wavelengths can produce a detectable difference in appearance (e.g., color and/or brightness) between a legitimate or authentic pharmaceutical product and a counterfeit or adulterated pharmaceutical product. Thus, the light sources selected for use in illuminating at least the suspect product is done so as to use light sources having wavelengths that are likely to produce a detectable difference in appearance (e.g., color and/or brightness) between a legitimate or authentic pharmaceutical product and a counterfeit pharmaceutical product. It should be noted that the object being illuminated is described as being a pharmaceutical product, however as mentioned previously other illuminated objects are within the inventive concepts which should not be construed to be limited only to pharmaceutical products and their packaging.

In some cases, the pharmaceutical product(s) are viewed by a user under the desired illumination (e.g., wavelength) through a filter, which can be incorporated into glasses or goggles 32 which filter out illumination wavelengths, and allow the wavelengths of the light or radiation returning from the illuminated product(s) to pass through. Although the different appearances of different products are not generally predictable a priori, with some experimentation, it has been found relatively easy to determine and document which illumination and filter wavelengths work well for distinguishing a given pharmaceutical product from a counterfeit version of that product.

Thus, it is within the scope of the present invention to establish criterion and operating protocols to follow that allow one to determine the type of light, the color of such light and any filtering requirements for viewing a suspect product to see if it is an authentic product. For example, it may be established from a series of experiments that, to determine whether an unknown tablet purportedly from Manufacturer A is counterfeit, the suspect product should be viewed under green light with a yellow filter, and in addition that when viewed in this manner, the color of the legitimate product has a bright yellow hue. Thus, when a suspect product when viewed under such conditions is a darker brownish and slightly red appearance, one can conclude that the suspect product is a counterfeit.

In yet further embodiments and for purposes of making the viewing more convenient and to potentially expand the observable emission spectrum, the detection device 20 further includes one or more image acquisition devices (e.g., cameras, CCD, night vision devices) 34a, 34b that are usable for imaging the pharmaceutical products under the desired illumination. In more particular embodiments, such image acquisition devices 34a, 34b are capable of detecting the light returning from the illuminated product or object and to provide an output representative of the detected light. For example, in a particular embodiment, an image acquisition devices 34a, 34b are configured and arranged so it detects light at or about a predetermined wavelength corresponding to a given color of light and provides an output representative of the detected brightness of the light.

In some embodiments, the one or more image acquisition devices 34a, 34b are capable of simultaneously imaging in a plurality of spectrums such as UV, visible light, and infrared. These images can also be transmitted and/or displayed simultaneously using the components discussed below. Also, the first and second imaging displays 36a, 36b may display the same image, but shown using different image acquisition devices. The different image acquisition devices may be adapted to receive or detect different wavelengths, or different bands of wavelengths from one another. As a non-limiting example a first image acquisition device may be configured to receive infrared light, which may be reflected off of the illuminated object, while the second image acquisition device may be configured to receive or detect visible and or ultraviolet light reflected off of the object. The first and second imaging displays may display the first and second images which were received/detected by the first and second image acquisition devices.

As described herein, such image acquisition devices 34a, 34b may be further configurable with a filter or the like so the returning light is filtered so that the light impinging upon the sensing component(s) of the image acquisition devices 34a, 34b may be at or about the given wavelength. In illustrative embodiments, such image acquisition devices 34a, 34b comprise any of a number of devices that are known to those skilled in the art including, but not limited to a CCD camera or the like.

As indicated herein, the light illuminating the suspect product and the authentic product includes non-visible radiation or light such as light in the UV and IR ranges that are outside the human visual spectrum. In such cases, the image acquisition device includes a device (e.g., night vision devices) that are sensitive to light or radiation having such wavelengths. This expands the range of light usable for illuminating the suspect and authentic product and thus expands the range over which differences in appearance can be exploited. As stated earlier the two separate imaging displays may simultaneously display images of the same object with different illumination for comparison to an authentic object which had been similarly illuminated and imaged. The imaging devices may further be used for detecting Stokes and anti-Stokes frequency shifting inks, dyes and other materials. These types of inks, dyes and materials can be used to authenticate different products.

In yet further embodiments, the image acquisition devices 34a, 34b is configured to include an optical adjustment capability, such that the image acquisition device is usable as a “portable microscope” by using macro zoom capabilities of the incorporated lens(es). A hand held up-close viewing of the objects being illuminated while using such macro lens, allows for high resolution viewing. When used in this manner, the device may include legs, e.g., at least three legs. The legs may have adjustable lengths and the legs may be adapted to screw into and out of the body of the device.

In further embodiments such a detection device 20 includes first and second displays 36a, 36b (e.g., LCD displays) that may be operably coupled to the control circuitry 26. In this way, when image data may be acquired by given image acquisition devices, the control circuitry may provide outputs to the displays to thereby cause the displays to provide images having a color and brightness that may be representative of the image data acquired or sensed by the given acquisition devices. It also is within the scope of the present invention for the control circuitry 26 to combine image data from one or more image acquisition devices so that the display reproduces a color image representative of the color that would be observed as if it were being viewed by the eye. In sum, the displays may be usable for visually displaying images of the pharmaceutical product(s) under the selected wavelength(s).

The detection device also is configurable with a memory 38 such as a memory (e.g., non-volatile or FLASH memory, an optical drive, or a solid state drive) to store information for the operation of the detection device, image data representative of one or more authentic pharmaceutical products or packaging. In more particular embodiments, such information includes instructions regarding the appropriate wavelengths to use for various products and previously acquired images of authentic and counterfeit pharmaceutical products. Such information is intended to allow an agent in the field or in situ to easily compare the appearance of suspected counterfeit to the authentic product.

For example, an image of an authentic product is stored in the memory 38 which is retrieved from the memory by the control circuitry 26. The image data is sent to the displays 34a, 34b so that the user can use the stored image as a reference image for comparison with the acquired image of the suspect product. The control circuitry 26 is configurable so that the stored image is displayed at least one of before or after the image of the suspect product is acquired. In further embodiments, the control circuitry 26 is configured so that both the stored image and the acquired image are displayed at the same time (e.g., side by side arrangement) much as would be seen if the detection device was illuminating the authentic and suspect products at the same time.

In yet further embodiments, the detection device 20 may include one or more communication devices or input/output devices 40 that allows communication between an external device such as for example a computer (e.g., personal computer) and the detection device. In this way, instructions, image data of authentic products or application program data/instructions can be downloaded to the detection device or previously acquired image data by others using such a device 20 either in a laboratory test environment or in the field can be downloaded to or from the detection device. Such an I/O device 40 includes a USB port or communication device, a network I/O device that allows communications over a wide area network (WAN) or a local area network (LAN) either using wireless or wired communication techniques, and/or a cellular transceiver adapted and configured to send and receive data over a cellular network (e.g., a network implanting the 3G or 4G standards).

Such a detection device 20 also may include a power source 100 that is operably coupled to the functionalities of the detection device and under the control of the control circuitry 26. Such a power source may include any of a number of sources of electrical power as is known to those skilled in the art and including for example rechargeable or non-rechargeable batteries (e.g., alkaline, lithium ion, metal hydride and the like) and capacitors or high power capacitors. Such power sources 100 also can further include any of a number of electrical functionalities known to those skilled in the art (e.g., transformers) so as to control the power (voltage, current) being outputted by the power source so as to be at or about an appropriate value. In more particular embodiments, the detection device 20 is constructed so as to made in a hand-held form and be portable. In further illustrative embodiments, the power source is a battery such as a 12 VDC portable battery, or is any center polarity power adapter (e.g., 12-15 VDC).

Referring now to FIG. 2B there is shown a detection device 20 according to another embodiment of the present invention. Reference shall be made to the discussion above regarding FIG. 2A for details of devices or functionalities having common reference numerals. In this embodiment, the detection device 200 includes two sections, a scanning section 210 and a power section 220 that includes a power source 100. The power section 220 is operably coupled to the scanning section 210 by a cable 230 so that the power source supplies the power to operate the scanning section. In this arrangement, the scanning section 210 is configured and constructed so as to be made in a hand-held form and be portable. Such a power section 220 need not be configured or made so as to be hand-held but can be configured so as to be portable or fixed so as to provide a larger power source. For example, in an illustrative embodiment, the power section 210 is belt mounted so as to be worn about the waist of the user.

The devices and methods described herein are particularly well suited for field work, such as that done by Customs agents at airports, inspection stations and other ports of entry into the United States. In particular, this is the case as the detection device 20 or scanning section 210 is configurable so as to be made in a hand-held form and portable.

Referring now to FIGS. 3A, B there is shown a representative example of a hand-held detection device according to the present disclosure. It should be recognized that is well within the skill of those knowledgeable in the art to configure the detection device as described in any of the embodiments described herein as well as configuring a scanning section 210 so as to embody feature shown and described herein in connection with FIGS. 3A-B.

There is shown in FIG. 3A, a top view of the detection device 20, which further includes a housing 50 in which various components of the device may be disposed. One such component are LCD display screens 52a, 52b, which display allows the user to view images acquired by cameras 70a, 70b or other image acquisition devices described further below. Although the display will be described herein as producing a visible spectrum output, it will be appreciated that other image processing techniques can be used to provide a visible depiction or display of non-visible UV and/or IR wavelengths emitted by the product under view that are detectable only by the camera, whereby detectable differences can be found outside normal human vision capabilities. Depending on the product, this can enhance the differences seen between a legitimate and counterfeit product when viewed on the LCD display.

Also disposed on and/or within the housing are ultraviolet, infrared and white light momentary push buttons 54a-k which turn on and off certain light sources on the bottom of the housing (discussed in reference to FIG. 3B below) corresponding to the buttons. The housing may also include an on-off switch 56 and an illumination intensity control 58 which is connected to a subset of, or possibly all of, the light sources. UV-Visible momentary push-buttons, which may turn on and off certain light sources on the bottom of the housing (FIG. 3B).

In yet further embodiments, the device may include video in and video out ports. Video output may interface with USB and power supply connections. Image capture or video capture may occur through the USB connection. As shown in FIG. 3A the device can include a number of other buttons and connectors, such as for example, connectors for interfacing with one or more external monitors or terminal, a menu button, a power interface and alarm, and a capture button 58.

In the device shown in FIG. 3A, one button controls a white light source used for normal light illumination, and other buttons may control LEDs having center wavelengths of about 351 nm, about 800 nm, about 900 nm, and about 1050 nm, respectively. It will be appreciated that such light sources may not be pure, but may emit in a wavelength band centered at or near the recited center wavelength. The bandwidth is not particularly critical to device function. It should be recognized that LED light sources with the recited center wavelengths and having suitable bandwidths are commercially available.

A “white” light source can contain a set of narrowband emissions at different locations in the visible spectrum or can have a flatter broadband emission spectrum across most or all of the visible range.

In addition, referring to FIG. 3A, UV-Visible momentary push buttons may control light sources having center wavelengths of about 525 nm, about 470 nm, about 455 nm, and about 405 nm, respectively. An illumination intensity control 58 may be provided to provide intensity adjustability for low or highly reflective surfaces.

Referring to FIG. 3B, the bottom of the housing, according to aspects, may include two high sensitivity CCD chips (cameras) 70a and 70b, either or both of which may contain a removable color lens filter, which, in one embodiment, is held in place by a rubber grommet, not shown. Although CCD chips are exemplified herein, the device can comprise any light sensitive device as are know to those skilled in the art such as photodiodes or the like. In further embodiments, the camera lenses are dismountable, high quality precision ground, multi-element glass micro-board lenses which results in chromatic aberration reduction. The device 20 may be usable with one or both color filters in place, or is usable without filters. As indicate herein, in some embodiments, the device does not comprise a display or an image acquisition device (e.g., CCD chip).

In more particular exemplary embodiments, the arrangement of the LEDs in FIG. 3B may be as follows: 351 nm, 405 nm, 455 nm, 470 nm, 525 nm, 800 nm, 900 nm, 1050 nm and white light. The 351 nm LEDs are generally of lower output power than the 405-800 nm LEDs. The 900 nm and 1050 nm LEDs emit light in the infrared region, while the 351 nm LEDs emit light in the ultraviolet region. These wavelengths are illustrative of a particular exemplary embodiment. Thus, it is within the scope of the present invention to utilize other wavelengths and/or wavelength combinations that are more appropriate for scanning and evaluating particular pharmaceutical and/or packaging or packaging components.

The detection device 20 including the control circuitry may be configurable to provide simultaneous multiple light source illumination capability for various specific analysis requirements. It will be appreciated that the arrangement of buttons/controls shown in FIG. 3A, and LEDs shown in FIG. 3B, are illustrative, and many variations of these can occur and are within the scope of the present invention. In addition, the invention also is not limited to the particular wavelengths mentioned above. Many variations of these can be used, and are also within the scope of the present invention.

The detection device 20 described herein may be ergonomically designed for hand-held comfort, is portable and lightweight, and fits inside a shirt or jacket pocket. Thus, it is well suited for work in the field, and obviates the need to send field samples to a laboratory for analysis. Thus, customs agents can quickly determine whether a suspect pharmaceutical is in fact counterfeit. If desired, the suspected counterfeit pharmaceutical can be subjected to further confirmatory testing using conventional methods.

Exemplary Methods of Detecting Counterfeit Pharmaceutical Products

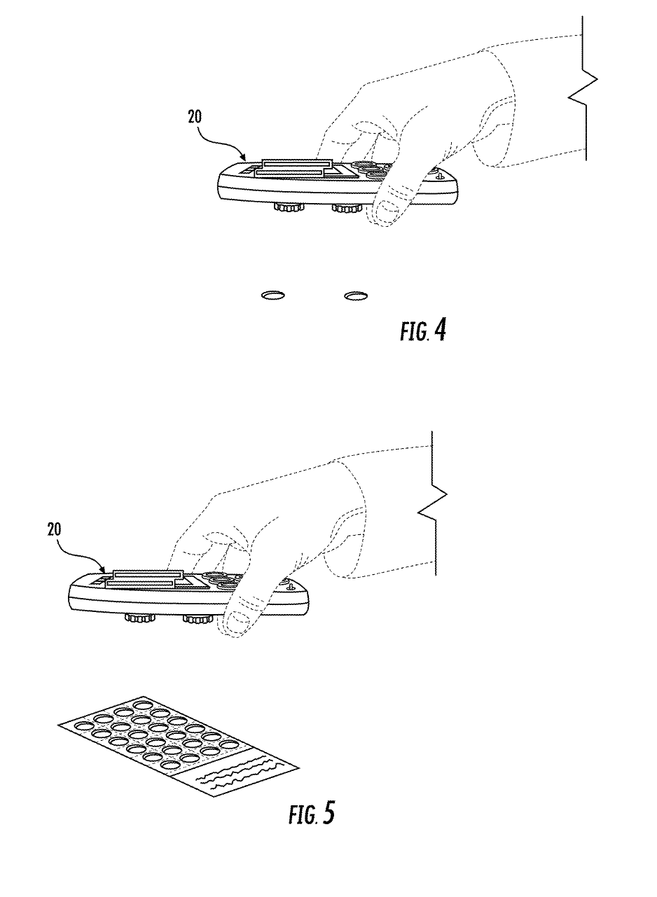

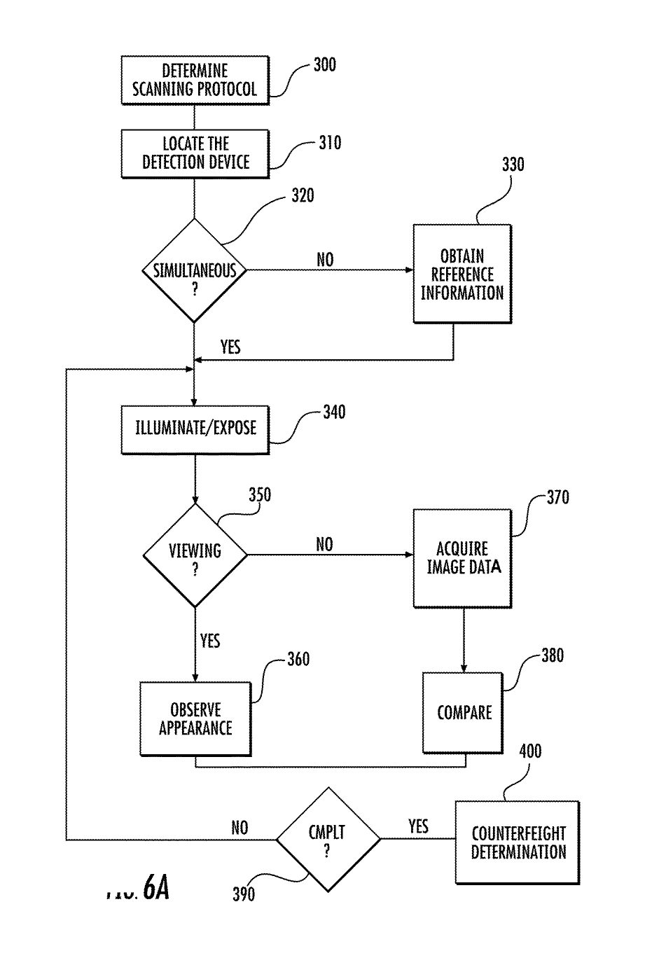

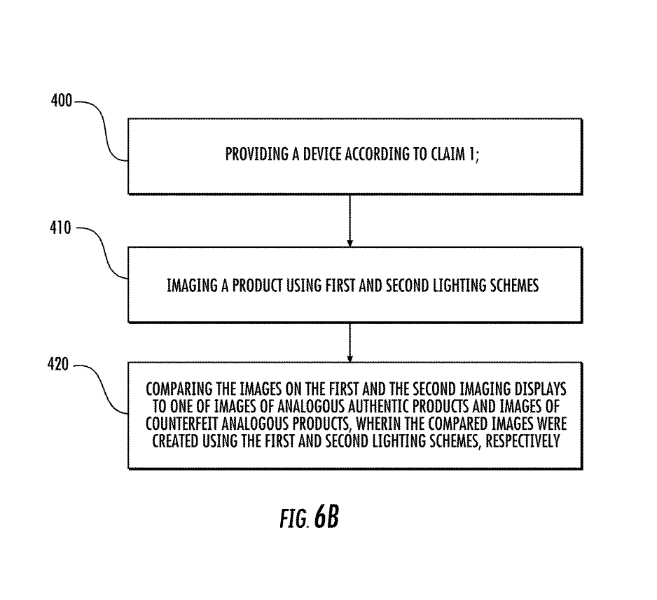

Now referring to FIGS. 4 and 5 which are diagrams showing the device in use for detection of suspected counterfeit pharmaceutical products both out of (FIG. 4) and in (FIG. 5) the packaging material and also to FIG. 6, which is a high level flow diagram of an illustrative embodiment of the methodology of the present invention. When a user is to conduct an examination of a suspect product or packaging, the user undertakes the steps necessary to establish the scanning protocol that will be used to determine if the suspect product is an authentic product or a counterfeit, Step 300. The user may initially determine the light sources having the wavelengths of light that should be used during such scanning to create the potential for determining from such a light scanning process if the suspect product is authentic or not. In addition, the user may determine if the light returning from the illuminated object(s) should be filtered or appropriately treated in conjunction with a direct viewing by the user or viewing via an image acquisition device. Further, the user may determine whether a light shield, shielding visible, ultraviolet, and/or infrared light should be used. As provided herein, in an embodiment of the present invention the suspect product or the suspect product and authentic product are viewed at the same time by a user that is wearing colored goggles or glasses.

If image acquisition devices are being utilized to acquire image data representative of the returning light (color and/or brightness) the user determines which of such devices should be used and in combination with what illuminating light sources. In an embodiment of the present invention, the user can operate a filter wheel to position a desired filter (e.g., colored filters such as orange or yellow, polarized, UV cutoff, and the like) over one or both camera lenses comprising the image acquisition devices. Thus, the user determines the appropriate filtering or is directed by the device as to which filter should be employed. It should be noted that the device may contain no camera, a single camera, two cameras, or three or more cameras, and that the presence of two cameras is only illustrative.

Also, the user can determine or be instructed as to whether the detection device should be oriented so as to be at an angle with respect to the object(s) being illuminated. For example, holding the detection device at oblique angles in some cases allow for better imaging/scanning analysis.

In sum, the user may determine at the outset the light sources, the light illumination sequencing, the image acquisition devices and other control parameters and the like that should be utilized to scan the suspect product/packaging and taking the appropriate steps so that scanning is done according to the determined protocol, which could be stored within the memory of the device.

After establishing the protocol and setting up the detection device, the user may locate the detection device in proximity to the suspect product, and in the case where the protocol includes simultaneously scanning the suspect and authentic product, locates the device in proximity to both of them, Step 310. For example and as shown in FIGS. 4-5, the detection device 20 may be held above the suspected counterfeit pharmaceutical product (FIG. 4) and/or product packaging (FIG. 5) by the user. In addition, the user may orient the detection device with respect to the object(s) to be illuminated in cases where better imaging and the like would be achievable.

If it is next determined if the process is proceeding with simultaneously viewing of the suspect and authentic product or not, Step 320. In the case where a suspected counterfeit pharmaceutical product and the corresponding authentic product (and/or product packaging) are placed side by side (Yes, Step 320), the suspect product and the authentic product are illuminated with the detection device 20 using one or more wavelengths of visible, ultraviolet or white light, Step 340 and differences in color and/or brightness of the authentic and suspected pharmaceutical products are observed or viewed by the user, Step 350. In an illustrative exemplary embodiment, the authentic and suspected pharmaceutical products and/or packaging are viewed under white light and light having a specific wavelength (e.g., 405, 455, 470 or 525 nm). As indicated herein, such viewing can be achieved by the user directly viewing the authentic and suspected pharmaceutical products and/or packaging while they are being illuminated and observing the appearance of both as they are being simultaneously illuminated. Alternatively, the appearance of the authentic and suspected pharmaceutical products and/or packaging are observed by viewing the appropriate LCD display screen 36.

The images of the two samples under the two different lighting conditions are then compared or the appearances of the samples are then compared, Step 360. Thereafter a determination is made whether or not the scanning protocol is completed, Step 390. If the process is not complete (No, Step 390), the process proceeds to illuminating or exposing at another set of wavelengths according to the protocol and steps 350 and 360 repeated as many times until it is determined that the process is complete (Yes, Step 390). If the process is determine to be complete, and if differences were observed from observing the appearance of the samples; such differences are evaluated to determine if they are representative of a suspected counterfeit, Step 400.

On the other hand, if it is next determined that the process is not proceeding with simultaneously viewing of the suspect and authentic product (No, Step 320), then the process proceeds with acquiring reference information that is representative of the authentic product, Step 330. For example, the control circuitry 26 retrieves information (acquired image data for the authentic product) from the memory 38 so it can be utilized later in the process. In this embodiment, the suspected counterfeit product is viewed alone, thus the suspected counterfeit product is illuminated with the detection device 20 using one or more wavelengths of visible, ultraviolet or white light, Step 340 and image data is acquired using the image acquisition devices, Step 370.

Thereafter, the acquired image data or image is then compared to the retrieved pre-existing image of the corresponding authentic product under the same illumination and detection conditions, Step 380. In this way, an agent in the field need not carry authentic samples of pharmaceutical products and or packaging with them as well as avoiding the need to take appropriate steps to maintain the authentic products so that they do not degrade, break down or otherwise become unusable as a reference sample.

In further embodiments the user would refer to the reference image one of before or after acquisition of the image for the suspect product and perform a comparison of the acquired image for the suspect product with reference image that was viewed before or after. In yet another embodiment and as provided herein, the control circuitry 26 controls the operation of the displays 36a, 36b so that the reference image and the acquired image of the suspect product are viewed simultaneously by the user. In other words, the two images are compared or the appearances of the images are compared.

Thereafter a determination is made whether or not the scanning protocol is completed, Step 390. If the process is not complete (No, Step 390), the process proceeds to illuminating or exposing the suspect product to another set of wavelengths according to the protocol and Steps 340, 370 and 380 are repeated as many times until it is determined that the process is complete (Yes, Step 390). If the process is determine to be complete, and if differences were observed from the performed comparison; such differences are evaluated to determine if they are representative of a suspected counterfeit, Step 400.

Another illustrative method for detecting a counterfeit product is illustrated in FIG. 6B and may include: providing a device according to claim 1, Step 400. A method may further include illuminating a product using first and second lighting schemes, Step 410. The first and second lighting schemes may include lighting using two specific wavelengths, two bands of wavelengths, etc. Such lighting schemes are more fully described herein in the description of the plurality of light sources 36a, 36b. The method may yet further include imaging, on each of the first and the second imaging displays, a product using first and second lighting schemes, respectively, Step 420 and comparing the images on the first and the second imaging displays to one of images of analogous authentic products and images of counterfeit analogous products, wherein the compared images were created using the first and second lighting schemes, respectively, Step 420.

According to aspects of the inventive concepts, methods may include storing an image, or a plurality of images of authentic products analogous to the products being illuminated for detection of counterfeits. Such stored images may be created using one or more lighting schemes as described herein above. For example, a pharmaceutical tablet may be imaged using ultraviolet light and an image of the authentic image may be stored in the device or external to the device. Once an analogous tablet is illuminated and imaged using the same ultraviolet lighting scheme, a user may compare the stored image to the newly acquired image.

While this method discloses visual comparison, it is within the scope of the inventive concepts to perform automated comparison of the stored image with the new image using any known, or yet-to-be developed imaging devices, and software, for example using spectroscopy, or other methods. A plurality of images of the authentic product, taken using a plurality of lighting schemes, may be stored for comparison, as well. And as discussed, the images may be stored in device 20 or external to device 20. One skilled in the art will appreciate that the comparison may be performed after the newly acquired image is transferred to a second device, as well. Further methods may include storing the images of the product/tablet for future use.

Although a preferred embodiment of the inventive concepts has been described using specific terms, such description is for illustrative purposes only, and it is to be understood that changes and variations may be made without departing from the spirit or scope of the following claims.

Detection device 20 can include a variety of components for imaging at various wavelengths. For example, the detection device can include one or more charge-coupled devices (CCDs) as discussed above. Illumination can be provided a plurality of light sources such as LEDs and/or white/Tungsten light. In one embodiment, the detection device 20 can produce nine distinct wavelengths (e.g., 365 nm, 375 nm, 405 nm, 455 nm, 470 nm, 535 nm, 630 nm, 667 nm, 850 nm, and 1050 nm) in addition to white/Tungsten light. Additionally or alternatively, the detection device 702 can also emit energy at a wavelength of 575 nm (i.e., yellow), which can be utilized for crime scene investigations. Detection device can include a rotary or a linear filtering device as described in U.S. Pat. No. 5,245,179 to selectively apply one or more filters over CCDs.

Detection device 20 can be configured to automatically capture a plurality of images illuminated and/or filtered at a various frequencies. Such a device is advantageously much quicker and easier to use than devices that require a user to identify which frequencies may be of interest and then manually select appropriately illumination and/or filtering frequencies. In one embodiment, the detection device 20 captures one or more images at every possible setting (i.e., every combination of illumination and filtering frequency), compares the resulting data to a database of known signatures for valid products, and alerts the user if the signature is not valid.

Electronic Control of LED Groups

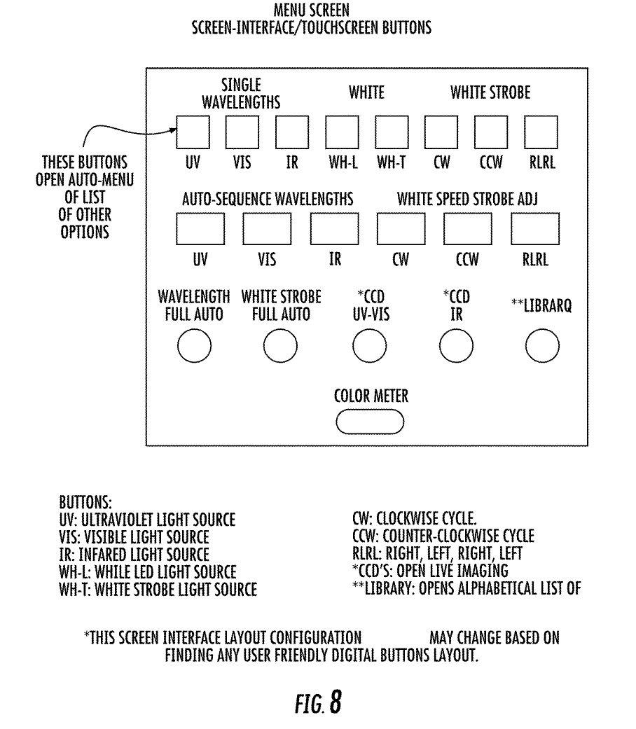

Referring now to FIG. 8, another aspect of the inventive concepts includes a plurality of screen interface touchscreen buttons. The buttons may be used operate distinct light sources individually, or in combination. The buttons may further operate the controller 26 to create strobe effects. The proposed circuitry in the device is designed for a multi-sequential directions of LED lighting, especially designed to suit a multipurpose application system. The circuit is integrated to the LEDs when a button is pressed, it is designed to reveal packaging features that are printed to display different colors based on different lighting angles. The circuit can also be modified to be an ordinary ON/OFF feature of many LEDs. The code written for these lighting options creates various chasing light patterns. The code can be easily modified and updated using any universal serial bus (USB) connections to various types of computing devices capable of modifying an Arduino type programing code.

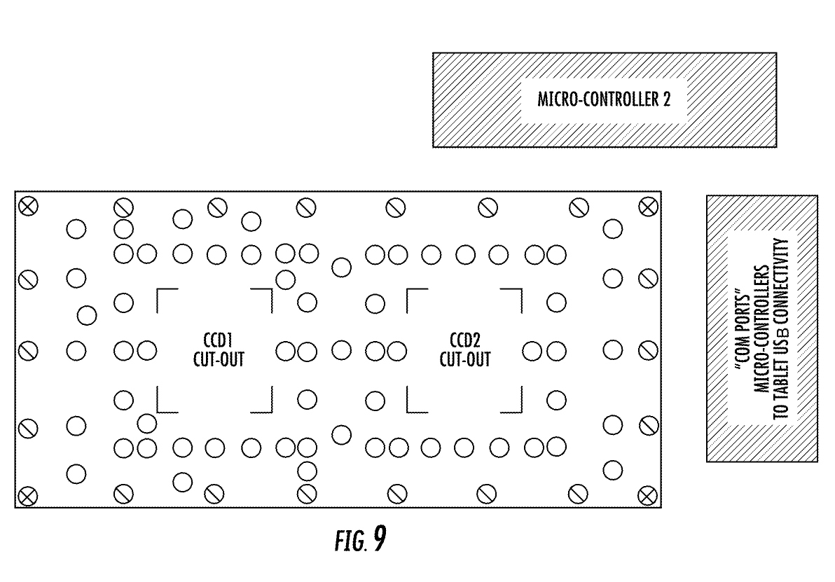

Referring now to FIG. 9, another aspect of the inventive concepts utilizes a relatively low-voltage microprocessor (e.g., an ARDUINO® microprocessor available from Arduinio, LLC of Cambridge, Mass.) to control relatively high voltage electronic components such as LEDs.

Instead of passing the relatively high voltage electricity through the microprocessor (which could damage or destroy the microprocessor), the microprocessor controls a transistor, which in turn, selectively completes a path from the LEDs to ground. Thus, the relatively high voltage does not pass through the microprocessor.

Illustrative Device Configurations

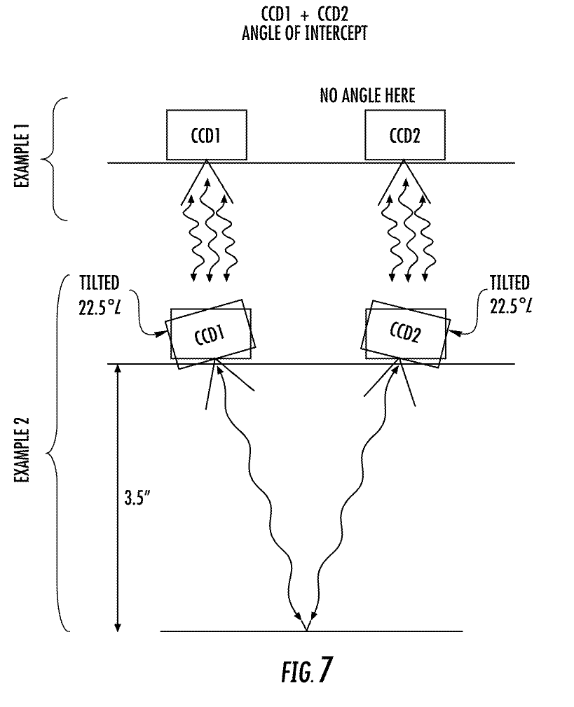

Referring now to FIG. 7, some embodiments of the presently disclosed devices tilt image acquisition devices (e.g., CCDs) with respect to a plan defined by the bottom of the device and/or the LEDs. For example, the image acquisition devices can be tilted about 22.5° with respect to the bottom of the device.

Tilted image acquisition devices provide several advantages. First, a tilted image acquisition device will receive more reflected energy from the product of interest. Second, tilted image acquisition devices can be aimed at the same location within a focal plane, thereby facilitating simultaneous imaging of the same location at different frequencies.

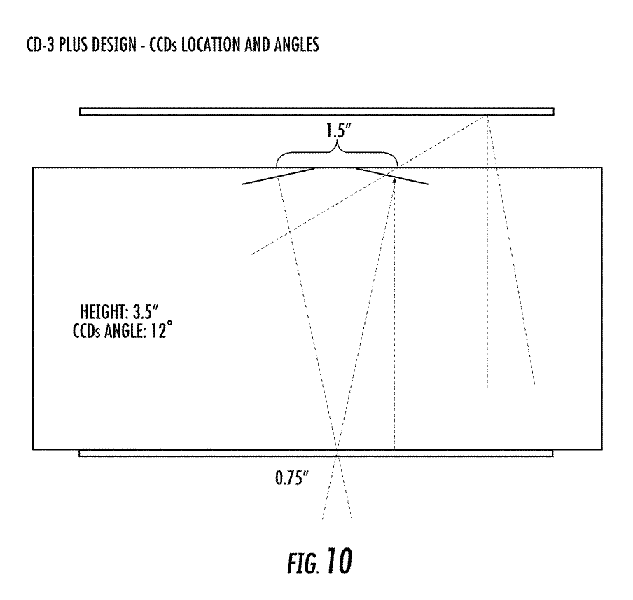

Referring now to FIG. 10, detection angles for CCDs and distances from illuminated objects according to an embodiment of the inventive concepts provided herein are depicted. An illustrative height above an illuminated object is 3.5 inches, however other heights are contemplated.

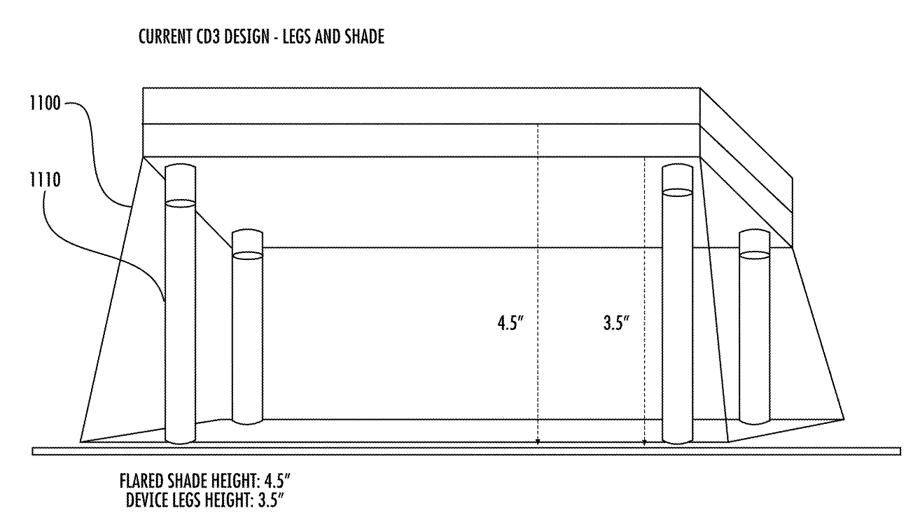

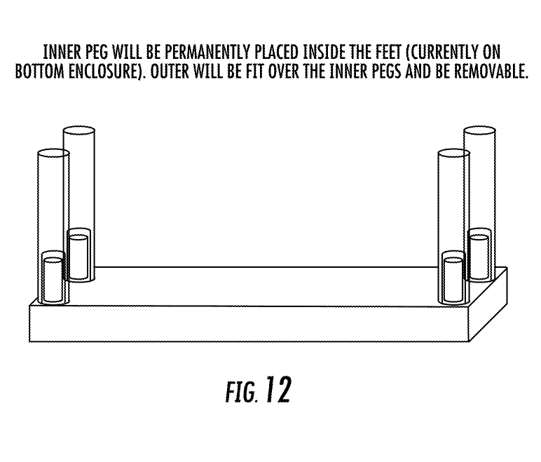

Referring now to FIGS. 11 and 12, some embodiments of the present disclosure include a shade 1100 and legs 1110. The legs may be adjustable in length and may be adapted to screw into and out of the body of the device 20 (FIG. 12). The shade 1100 may be made of any suitable material to prevent ambient light of different wavelengths to illuminate the object. For example shade 1110 may prevent ambient visible light, UV, and/or infrared from illuminating the object.

An example of this focusing can be seen in FIG. 11, which depicts an embodiment of the device including a pair of legs that can be extended and contracted (not shown) to allow the device to stand on a table, desk, counter, or other surface. The legs may be of adjustable length, and while not illustrated, the legs may be adapted to screw into and out of the body of the device.

### Field Screening Example

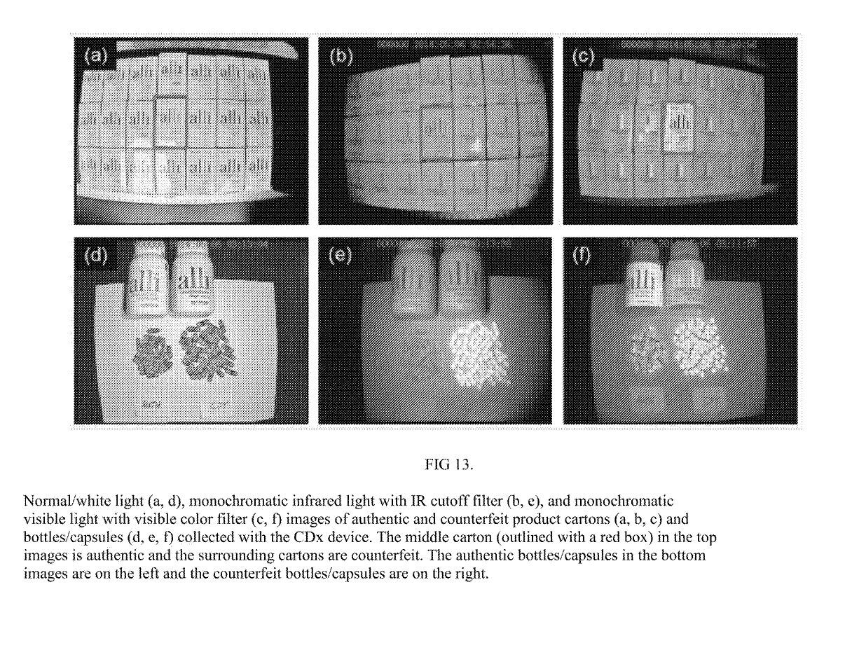

Referring now to FIG. 13 which provides a series of photographs illustrating a field screening using a detection device of the present disclosure of counterfeit and authentic product cartons, bottles and capsules. Large sample sizes encountered in the field and/or large sample submissions received by the laboratory can be screened initially using normal/white light and an alternate light source (ALS). For example, a detection device constructed in accordance with an embodiment of the present disclosure was used to collect the images shown in FIG. 13. Based on the white light image in FIG. 13a, the suspect cartons appear similar to those of the authentic (outlined by the bold box). However, clear differences are observed between the suspect and authentic cartons in the ALS images. In the infrared image provided in FIG. 13b, “alli” is clearly visible on the authentic carton whereas the “a,” first “l,” and “i” are all invisible on each of the suspect cartons.

In FIG. 13c, the authentic paper board is brighter and “alli” is clearly visible using a monochromatic wavelength of visible light. On the other hand, the suspect paperboard is darker, the “a” and “i” in “alli” are nearly invisible, and the second “l” is much brighter than that of the authentic. Regarding the bottles and capsules, the suspect products (right) appear visually consistent with those of the authentic (left) based on the white light image provided in FIG. 13d. While the suspect capsules are much brighter than those of the authentic based on the infrared image provided in FIG. 13e, few differences were observed between the suspect and authentic bottle labels. However, when examined using monochromatic visible light image in FIG. 13f, both the suspect and authentic bottles and capsules can be easily differentiated; the suspect label is darker, the suspect label printing is brighter, and the suspect capsules exhibit stronger fluorescence. This latter example demonstrates the importance of using more than 1 illumination wavelength when using ALS to compare suspect and authentic products.

In general, ALS examinations using a detection device of the present disclosure take less than 1 minute per sample to make an acceptance/rejection decision (ie determining if the sample needs to be sent to the laboratory for further analysis).

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A device, comprising:
a plurality of light sources configured to emit light at a plurality of different wavelengths onto an object;
at least two image acquisition devices adapted to acquire first and second image data; and
first and second imaging displays configured to display the first and the second image data, respectively.

2. The device of claim 1, wherein the plurality of light sources include light emitting diodes.

3. The device of claim 2, wherein the light emitting diodes includes single-wavelength light emitting diodes.

4. The device of claim 1, wherein the first image acquisition device is adapted to detect near-infra-red light and the second image acquisition device is adapted to detect light in the visible to ultraviolet spectrum.

5. The device of claim 1 further comprising a housing, wherein the plurality of light sources and the at least two image acquisition devices are coupled to the housing so that the plurality of light sources and the at least two image acquisition devices are maintained in fixed relation to each other while the object is being illuminated.

6. The device of claim 1, further comprising a control device adapted and configured to:
control operation of the plurality of light sources and the at least two image acquisition device; and
cause white light sources to flash sequentially.

7. (canceled)

8. The device of claim 1, further comprising one or more filters adapted to selectively condition light entering the at least one image acquisition device.

9. (canceled)

10. The device of claim 1, wherein at least one of the light sources of the plurality of light sources is adapted to emit light having an infrared wavelength, at least one of the light sources of the plurality of light sources is configured to emit light having a visible wavelength, and at least one of the light sources of the plurality of light sources is configured to emit light having an ultraviolet wavelength.

11. (canceled)

12. (canceled)

13. The device of claim 1, wherein said light sources include tungsten light sources.

14. The device of claim 5, further comprising at least three legs associated with the housing and adapted to allow for stationary operation of the device.

15. The device of claim 14, wherein the legs are adapted to have adjustable lengths.

16. The device of claim 15, wherein the legs are adapted to twist on and off.

17. The device of claim 1, wherein the plurality of light sources includes at least 12 light sources, each light source adapted to emit light of a wavelength different from the wavelengths emitted by other light sources.

18. The device of claim 5, wherein the housing further comprises a light shield adapted to prevent stray light from illuminating the object.

19. The device of claim 19, wherein the shield is adapted to block infrared light, and wherein the shield includes at least one magnet adapted to magnetically attach the shield to of the housing of the device.

20. (canceled)

21. (canceled)

22. A method, comprising the steps of:
providing a device according to claim 1;
illuminating, by the first and second light sources of the plurality of light sources, a product using first and second lighting schemes;
imaging, on each of the first and the second imaging displays, the product; and
comparing the images on the first and the second imaging displays to one of images of analogous authentic products and images of counterfeit analogous products, wherein the compared images were created using the first and second lighting schemes, respectively.

23. The method of claim 22, further comprising:
prior to the comparing of the images on the first and the second imaging displays, storing an image of an authentic product in a memory of the device, wherein the image of the authentic product is associated with one of the first or the second lighting schemes.

24. The device of claim 1, wherein the object is a pharmaceutical product.

25. The device of claim 24, wherein the pharmaceutical product is a pharmaceutical tablet or capsule, or pharmaceutical product packaging.

26. The device of claim 1, wherein the object is a food product.

27. The method of claim 22, wherein the product is a pharmaceutical product.

28. The method of claim 27, wherein the pharmaceutical product is a pharmaceutical tablet or capsule, or pharmaceutical product packaging.

29. The method of claim 22, wherein the product is a food product.

30. The method of claim 22, wherein the method is a method of detecting a counterfeit product.
